SEQUENCE LISTING (10 sequences)

SEQ ID NO: 1 - DNA, 50 bp, Danio rerio
gtctcaaatttgcggcctttctaagaatgtcagattatggcttgatcgaa

SEQ ID NO: 2 - DNA, 50 bp, Danio rerio
ttcgatcaagccataatctgacattcttagaaaggccgcaaatttgagac

SEQ ID NO: 3 - DNA, 20 bp, Artificial Sequence
  Synthetic Polynucleotide
tttctaagaatgtcagatta

SEQ ID NO: 4 - DNA, 20 bp, Artificial Sequence
  Synthetic Polynucleotide
ataatctgacattcttagaa

SEQ ID NO: 5 - DNA, 50 bp, Danio rerio
cgatgaggaatgggaataactggcctcacacctgttatatttattttatt

SEQ ID NO: 6 - DNA, 50 bp, Danio rerio
aataaaataaatataacaggtgtgaggccagttattcccattcctcatcg

SEQ ID NO: 7 - DNA, 20 bp, Artificial Sequence
  Synthetic Polypeptide
aaataaatataacaggtgtg

SEQ ID NO: 8 - DNA, 50 bp, Danio rerio
gccaaatcaacatggtgaagcggacattgatgctccggtagatttgaaga

SEQ ID NO: 9 - DNA, 50 bp, Danio rerio
tcttcaaatctaccggagcatcaatgtccgcttcaccatgttgatttggc

SEQ ID NO: 10 - DNA, 20 bp, Artificial Sequence
  Synthetic Polynucleotide
tgaagcggacattgatgctc

(19) United States
(12) Patent Application Publication
(10) Pub. No.: US 2022/0015341 A1
(43) Pub. Date: Jan. 20, 2022

(54) STERILE FISH

(71) Applicants: Recombinetics, Inc., Eagan, MN (US); Mayo Foundation for Medical Education and Research, Rochester, MN (US)

(72) Inventors: Karl J. Clark, Rochester, MN (US); Tad S. Sonstegard, Centreville, MD (US); Daniel F. Carlson, Woodbury, MN (US)

(21) Appl. No.: 17/496,721

(22) Filed: Oct. 7, 2021

Related U.S. Application Data
(63) Continuation of application No. PCT/US2020/027560, filed on Apr. 9, 2020.
(60) Provisional application No. 62/831,293, filed on Apr. 9, 2019.

(51) Int. Cl. A01K 67/027; C12N 9/22; C12N 15/90; C12N 15/02

(52) U.S. Cl. CPC A01K 67/0276; C12N 9/22; C12N 15/902; C12N 2310/20; A01K 2217/075; A01K 2227/40; C12N 15/02

(57) ABSTRACT

The present disclosure provides, at least, sterile fish and methods for producing sterile fish.

### CROSS-REFERENCE TO RELATED APPLICATIONS

This is a continuation application of International Application No. PCT/US2020/027560, filed Apr. 9, 2020, which application claims priority to U.S. 62/831,293, filed Apr. 9, 2019, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCI copy, created on Oct. 7, 2021, is 2,008 bytes in size and is named 53545_731_301_SequenceListing_ST25.txt.

### BACKGROUND OF THE INVENTION

If a farmed fish having improved traits escapes, it can transmit its improved genes into wild populations of fish. Such an improved fish may outcompete its wild relatives. To avoid this, it is desirable to create improved fish that are sterile, such that if a farmed fish escapes, it is unable to transmit its improved genes into wild populations of fish and, possibly, outcompete the wild populations of fish, thereby reducing diversity in the wild.

### SUMMARY OF THE INVENTION

The present disclosure provides, at least, sterile fish and methods for producing sterile fish.

In an aspect, the present disclosure provides a method for producing a sterile fish. The method includes a step of fertilizing an egg with a sperm, wherein the egg is obtained from a female fish comprising a gene-edited homozygous alteration in the three prime untranslated region (3′-UTR) of a gene.

The alteration in the 3′-UTR of the gene may results in a dysfunction in a maternally-expressed mRNA. The maternally-expressed mRNA that comprises the dysfunction may be deposited into the egg by the female fish comprising the homozygous alteration. In embodiments, the dysfunction in the maternally-expressed mRNA prevents or reduces development and/or migration of primordial germ cells (PGCs) in the fertilized egg, in a resulting zygote, and/or in a resulting larva.

The sterile fish may produce a reduced number of gametes relative to a fish resulting from fertilization of an egg obtained from a female fish lacking the homozygous alteration. In embodiments, the sterile fish fails to produce any gametes. The sterile fish may produce a reduced number of functional gametes relative to a fish resulting from fertilization of an egg obtained from a female fish lacking the homozygous alteration. In embodiments, the sterile fish fails to produce any functional gametes.

The fertilizing may be in vitro or the fertilizing may be in vivo and comprises mating a male fish and the female fish comprising the homozygous alteration. In the latter case, the mating may comprise internal fertilization of the egg or external fertilization of the egg.

In some cases, the method further comprises maintaining the fertilized egg, the resulting zygote, and/or the resulting larva under conditions suitable for development of the sterile fish into a fry the method further comprises maintaining the fry under conditions suitable for development of the sterile fish into a juvenile, and/or the method further comprises maintaining the juvenile under conditions suitable for development of the sterile fish into a fully grown, mature, and/or adult fish.

In embodiments, the sterile fish is male.

The sperm used in fertilization may comprise an alteration in the 3′-UTR of the gene or the sperm may lack an alteration the 3′-UTR of the gene, i.e., may not have a gene-edited 3′-UTR.

In embodiments, the gene (when not gene-edited in its 3′-UTR) contributes to normal development and/or normal migration of primordial germ cells. The gene may be one or more of nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, and puf/puf-A.

The alteration (in the 3′-UTR of the gene) may have been gene-edited in an unfertilized egg or in a fertilized egg. In embodiments, the egg that was gene-edited was obtained from a progenitor of the female fish comprising the homozygous alteration.

The alteration (in the 3′-UTR of the gene) may have been gene-edited in a zygote. In embodiments, the egg that was gene-edited was obtained from a progenitor of the female fish comprising the homozygous alteration.

In some cases, the method further comprises obtaining a cell's nucleus comprising the alteration in the 3′-UTR of the gene. In embodiments, the method further comprises transferring the nucleus comprising the alteration to an enucleated egg; the enucleated egg receiving the nucleus develops into the progenitor of the female fish comprising the homozygous alteration. The alteration in the 3′-UTR of the gene may have been gene-edited in the cell providing the nucleus or was gene-edited in a parent cell.

The gene-editing steps may employ one or more of microinjection, lipid-based transfection, chemical-based transfection, electroporation, viral-mediated transduction, or exosome-mediated transfected, and a combination thereof. In embodiments, the micronuclear injection is pronuclear microinjection. In embodiments, the lipid-based transfection comprises nanoparticles, microparticles, or liposomes, and a combination thereof.

In some cases, the progenitor precedes the female fish (that comprises a gene-edited homozygous alteration in the 3′-UTR of a gene) by at least one generation, at least two generations, at least three generations, at least five generations, at least ten generations, or at least one hundred generations, and any number of generations therebetween.

In embodiments, the gene-editing comprises use of a nuclease.

In some cases, the gene-editing comprises a site-specific gene editing system. The gene editing system may comprise CRISPR/CaS, a TALEN, a zinc finger nuclease, or a meganuclease.

In embodiments, the gene editing system creates an alteration that comprises a deletion in the 3′-UTR, e.g., a deletion which results in a premature truncation of the 3′-UTR. In embodiments, the deletion prevents normal development and/or normal migration of primordial germ cells and/or reduces or abolishes recognition of the 3′-UTR by its binding protein. The gene editing system may create an alteration that comprises an insertion of a nucleic acid sequence into the 3′-UTR.

The gene-editing may comprise a polynucleotide.

In embodiments, the polynucleotide comprises one or more regions homologous to the gene's 3′-UTR nucleotide sequence and/or the polynucleotide may comprise one or more regions non-homologous to the gene's 3′-UTR nucleotide sequence. In embodiments, the polynucleotide comprises a homology directed repair (HDR) template or the polynucleotide comprises a guide RNA (gRNA). In embodiments, the gene-editing comprises a polynucleotide and a guide RNA (gRNA).

In embodiments, a non-homologous region comprises a sequence that prevents normal development and/or normal migration of primordial germ cells. The sequence that prevents normal development and/or normal migration of primordial germ cells may reduce or abolish recognition of the 3′-UTR by its binding protein and/or may comprise one or more additional nucleotides. In some cases, the sequence that prevents normal development and/or normal migration of primordial germ cells comprises a coding sequence for an exogenous gene. In embodiments, the coding sequence for the exogenous gene comprises a promoter, e.g., a constitutive promoter or a tissue-specific promoter. In embodiments, the exogenous gene encodes a reporter, e.g., a fluorescent protein. The fluorescent protein may be a derivative or variant of green-fluorescent protein (GFP).

The sterile fish may be tilapia (e.g., Mozambique tilapia (Oreochromis mossambicus) and Nile tilapia (Oreochromis niloticus); salmon (e.g., Atlantic salmon (Salmo salar), Chinook salmon (Oncorhynchus tshawytscha), and Coho salmon (Oncorhynchus kisutch)); trout (e.g., Rainbow trout (Oncorhynchus mykiss*); tuna (e.g., Bluefin Tuna (Thunnus thynnus); seabass (e.g., European seabass (Dicentrarchus labrax); bream (e.g., White amur bream (Parabramis pekinensis); seabream (e.g., Red seabream* (Pagrus major*); barramundi* (Lates calcarifer); milkfish (Chanos chanos); catla (Catla catla*); carp (e.g., Crucian carp (Carassius carassius), Mud carp (Cirrhinus molitorella*), Mrigal carp (Cirrhinus mrigala), Grass carp (Ctenopharyngodon idellus), Common carp (Cyprinus carpio), Silver carp (Hypophthalmichthys molitrix), Bighead carp (Hypophthalmichthys nobilis*), Roho labeo (Labeo rohita), Black carp (Mylopharyngodon piceus)); catfish (e.g., Channel catfish (Ictalurus punctatus)); amberjack (e.g., Japanese amberjack (Seriola quinqueradiata); or zebrafish (Danio rerio).

The female fish comprising a gene-edited homozygous alteration and/or the progenitor may further comprise an improved trait relative to a wild-type fish of similar species. The improved trait may be the result of genetic engineering and/or the result of selective breeding. The improved trait may any trait that has been introduced or bred into fish and that enhances the value of a commercial fish. The improved trait may be one or more of area of fat depot, body shape, disease resistance, faster growth, fat percentage, flesh color, greater protein content, improved fertility, larger muscles, skin color, and temperature tolerance.

In an aspect, the present disclosure provides a sterile fish obtained by any herein-disclosed method.

In another aspect, the present disclosure provides a food product comprising tissue obtained from the sterile fish obtained by any herein-disclosed method.

An aspect of the present disclosure is an in vitro cell. The in vitro cell comprises a heterozygous alteration in the three prime untranslated region (3′-UTR) of a gene or a homozygous alteration in the 3′-UTR of a gene. In these aspects, the gene contributes to normal development and/or normal migration of primordial germ cells.

In embodiments, the in vitro cell is a somatic cell, an unfertilized egg, a fertilized egg, or a sperm cell.

Another aspect of the present disclosure is an in vivo cell. The in vivo cell comprises a heterozygous alteration in the three prime untranslated region (3′-UTR) of a gene or a homozygous alteration in the 3′-UTR of a gene. In these aspects, the gene contributes to normal development and/or normal migration of primordial germ cells.

In embodiments, the in vivo cell is a somatic cell, an unfertilized egg, a fertilized egg, or a sperm cell.

Another aspect of the present disclosure is a fish comprising a heterozygous alteration in the three prime untranslated region (3′-UTR) of a gene.

Yet another aspect of the present disclosure is a fish comprising a homozygous alteration in the three prime untranslated region (3′-UTR) of a gene.

In these aspects, the gene contributes to normal development and/or normal migration of primordial germ cells. The gene may be nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, or puf/puf-A.

In some cases, the alteration in the 3′-UTR of the gene reduces or abolishes recognition of the 3′-UTR by its binding protein. In embodiments, the alteration comprises a deletion in the 3′-UTR, e.g., a premature truncation of the 3′-UTR. In embodiments, the deletion prevents normal development and/or normal migration of primordial germ cells. In embodiments, the alteration comprises an insertion of a nucleic acid sequence into the 3′-UTR, e.g., a nucleic acid sequence that comprises a coding sequence for an exogenous gene. The exogenous gene may encode a reporter. In embodiments, the insertion prevents normal development and/or normal migration of primordial germ cells.

The fish may be tilapia (e.g., Mozambique tilapia (Oreochromis mossambicus) and Nile tilapia (Oreochromis niloticus); salmon (e.g., Atlantic salmon (Salmo salar), Chinook salmon (Oncorhynchus tshawytscha), and Coho salmon (Oncorhynchus kisutch)); trout (e.g., Rainbow trout (Oncorhynchus mykiss*); tuna (e.g., Bluefin Tuna (Thunnus thynnus); seabass (e.g., European seabass (Dicentrarchus labrax); bream (e.g., White amur bream (Parabramis pekinensis); seabream (e.g., Red seabream* (Pagrus major*); barramundi* (Lates calcarifer); milkfish (Chanos chanos); catla (Catla catla*); carp (e.g., Crucian carp (Carassius carassius), Mud carp (Cirrhinus molitorella*), Mrigal carp (Cirrhinus mrigala), Grass carp (Ctenopharyngodon idellus), Common carp (Cyprinus carpio), Silver carp (Hypophthalmichthys molitrix), Bighead carp (Hypophthalmichthys nobilis*), Roho labeo (Labeo rohita), Black carp (Mylopharyngodon piceus)); catfish (e.g., Channel catfish (Ictalurus punctatus)); amberjack (e.g., Japanese amberjack (Seriola quinqueradiata); or zebrafish (Danio rerio).

In embodiments, the fish is female.

In some cases, the fish further comprises an improved trait relative to a wild-type fish of similar species. The improved trait may be the result of genetic engineering and/or the improved trait is the result of selective breeding. The improved trait may any trait that has been introduced or bred into fish and that enhances the value of a commercial fish.

The improved trait may be one or more of area of fat depot, body shape, disease resistance, faster growth, fat percentage, flesh color, greater protein content, improved fertility, larger muscles, skin color, and temperature tolerance.

A final aspect of the present disclosure is a method to rescue the sterility in a fish resulting from a gene-edited alteration in the three prime untranslated region (3′-UTR) of a gene responsible for germ plasm migration and/or gamete development. The method comprises injecting into an egg from the fish a wild type copy of mRNA corresponding to the gene that comprises gene-edited alteration.

In embodiments, the offspring of the egg would be fertile but the offspring produces sterile offspring.

Any aspect or embodiment described herein can be combined with any other aspect or embodiment as disclosed herein.

### BRIEF DESCRIPTION OF THE DRAWINGS

Various illustrative aspects and embodiments of the sterile fish of the present invention and methods related thereto will be described in detail, with reference to the following figures.

FIG. 1A to FIG. 1D are illustrations showing germ plasm migration in fish zygotes. Germ plasm refers to cytoplasmic components (including specific proteins and mRNAs) present in zygotes/early embryos that help drive determination of the primordial germ cells, which ultimately form into gametes in an adult fish.

FIG. 2 shows further development of PGCs in fish embryos and in larva.

FIG. 3 is a schematic illustrating steps for genetic engineering (including gene-editing) fish.

FIG. 4 is a schematic illustrating steps in a breeding program for producing sterile males.

FIG. 5 illustrates a breeding program for expanding fertile females comprising homozygous alterations.

FIG. 6 illustrates steps in Embryonic Rescue of Sterility.

FIG. 7A to FIG. 7C, respectively, show the structure of the dnd1/dnd, ddx4/vasa, and nanos3/nanos/nanos1 genes in Danio rerio. In FIG. 7A, the upper and lower DNA strands show a portion of the dnd1/dnd3′-UTR which have sequences covered by SEQ ID NO: 1 and SEQ ID NO: 2 and the sG1 sequence is covered by SEQ ID NO: 3 and the sG4 sequence is covered by SEQ ID NO: 4. The sG1 and sG4 sequences are targets sites for illustrative gRNAs. In FIG. 7B, the upper and lower DNA strands show a portion of the ddx4/vasa 3′-UTR which have sequences covered by SEQ ID NO: 5 and SEQ ID NO: 6 and the sG1 sequence is covered by SEQ ID NO: 7, which is a target site for an illustrative gRNA. In FIG. 7C, the upper and lower DNA strands show a portion of the nanos3/nanos/nanos1 3′-UTR which have sequences covered by SEQ ID NO: 8 and SEQ ID NO: 9 and the sG1 sequence is covered by SEQ ID NO: 10, which is a target site for an illustrative gRNA.

FIG. 8 shows an illustrative polynucleotide that may be used to replace the wild-type 3′-UTR of a gene relevant to the development, maturation, and/or migration of PGCs.

FIG. 9A and FIG. 9B show dnd1^{+/g1STOP} in-cross fish that is injected with GFP nanos3′-UTR marker construct mRNA. GPF-positive punctate mark PGCs; the GFP also labeled retinas.

FIG. 10A and FIG. 10B show ddx4/vasa^{-gRNA1} F2 fish from a het in-cross of F1 parents that is injected with GFP nanos3′-UTR marker construct mRNA. GPF-positive punctate mark PGCs; the GFP also labeled retinas.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### DETAILED DESCRIPTION OF THE INVENTION

The present disclosure is based, in part, on the discovery methods for producing sterile fish.

### Introduction

So far, nearly fifty species of fish have been genetically engineered; these include trout, catfish, tilapia, striped bass, flounder, and many species of salmon. These fish are being genetically engineered for improved traits that will make them better suited, at least, for industrial aquaculture, such as faster growth, disease resistance, larger muscles, and temperature tolerance. Transgenic fish have also been specifically created to act as bio-indicators and bio-reactors. It is expected that transgenic methods will be used to produce gametes from endangered species, such as yellowtail (Seriola quinqueradiata) and other fish species that have suffered from over-fishing. See, Tonelli et al, “Progress and biotechnological prospects in fish transgenesis.” Biotechnology Advances Volume 35, Issue 6, 1 Nov. 2017, Pages 832-844.

In 2015, FDA approved a genetically engineered salmon (the AquAdvantage salmon) as fit for human consumption. This made the AquAdvantage salmon the first genetically altered animal to be cleared for American supermarkets and dinner tables. The AquAdvantage salmon is an Atlantic salmon that has been genetically modified so that it grows to market size faster than a non-engineered farmed salmon, in as little as half the time. This fish, like other transgenic fish, can provide the market with a great supply of sustainably-farmed fish products.

However, there is a concern that should a genetically-engineered fish that comprises improved traits (in particular, a shortened time to sexual maturity) escape from a farm, it could breed with and outcompete wild fish. In a recent study, researchers concluded that if genetically engineered male Atlantic salmon were to escape, they could succeed in breeding and passing their genes into the wild. Furthermore, such escaped genetically engineered fish likely would pose a serious threat in reducing biodiversity. It has been estimated that genetically-engineered fish that breed with wild populations, could lead to the extinction of the wild population in tens of generations. This concern, in part, has led the US states of Washington and Maine to imposed permanent bans on the production of transgenic fish.

However, these concerns that fish with improved traits that escape from a farm and outcompete wild fish is not limited to genetically-engineered fish. Instead, this concern is also relevant for fish having improved traits due to conventional selective breeding. The improved traits in fish that were selected for in the farm (e.g., greater protein content, rapid development, and thermotolerance) may also confer advantages for the farmed fish when escaped into the wild.

Moreover, fish that are not native to a region may lack natural predators, may newly prey on the native species, and/or may be especially well-suited for the region. Such non-native fish likely will have a competitive advantage once escaped from the farm. There are numerous examples of intentional, and well-intentioned, stocking of waterways with non-native fish that later became invasive species, and which decimated native populations of fish.

The present disclosure provides sterile fish, and methods for producing the same. These sterile fish, if escaped from a farm, would be unable to breed with wild populations and would be unable to pose a serious threat in reducing native biodiversity.

### Methods for Producing a Sterile Fish

In one aspect, the present disclosure provides a method for producing a sterile fish. The method includes a step of fertilizing an egg with a sperm, wherein the egg is obtained from a female fish comprising a gene-edited homozygous alteration in the three prime untranslated region (3′-UTR) of a gene.

The three-prime untranslated region (3′-UTR) is the section of a gene or the encoded messenger RNA (mRNA) that immediately follows the translation termination codon. The 3′-UTR often contains regulatory regions that post-transcriptionally influence gene expression. The 3′-UTR may play a crucial role in gene expression by influencing the localization, stability, export, and translation efficiency of an mRNA. Some 3′-UTR comprise sequences that are involved in gene expression, including microRNA response elements (MREs), AU-rich elements (AREs), and the poly(A) tail.

All sexually reproducing organisms arise from the fusion of gametes—sperm and eggs. All gametes arise from the primordial germ cells. In many animal species, the determination of the primordial germ cells is brought about by the cytoplasmic localization of specific proteins and mRNAs in certain cells of the early embryo. These cytoplasmic components are referred to as the germ plasm.

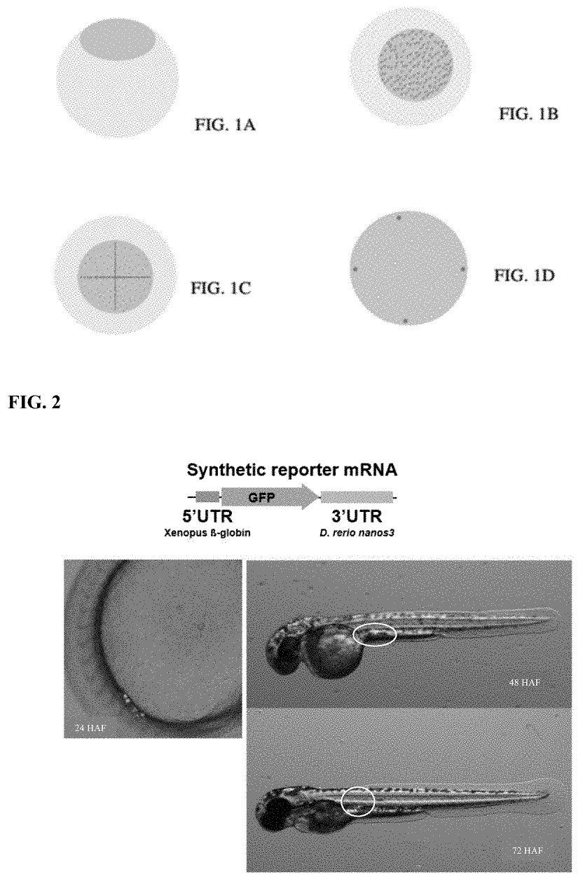

FIG. 1A to FIG. 1D illustrate the process of germ plasm migration and typical primordial germ cell (PGC) development in the fish zygote/embryo. FIG. 1A is a side-view of an egg (unfertilized or fertilized) or a one-cell zygote in which the germ plasm coalesces at the top of the zygote. FIG. 1B shows a top view of the zygote where the maternally deposited germ plasm mRNA is deposited evenly along with the cytoplasm around the yolk and coalesces with the cytoplasm to form a single cell. In some instances, maternal genes are transcribed into mRNAs by the mother and deposited by her into an egg. FIG. 1C illustrates the migration of germ plasm at about the 4-cell stage where the mRNA begins to become localized at the cleavage furrows. The germ plasm that has not migrated to the cleavage furrows begins to degrade shortly thereafter. During subsequent cell divisions, the four tight germ plasm structures segregate asymmetrically between the dividing cells thus, maintaining the number of germ plasm containing cells constant. At the embryo's sphere stage, the germ plasm appears to spread in the cytoplasm and after cell division, it is inherited by both cells leading to an increase in the number of the PGCs. Considering that the orientation of the cleavage planes in zebrafish is random relative to the future dorsal aspect of the embryo, the four PGC clusters too, are found in random positions relative to the dorsal aspect of the embryo. FIG. 1D illustrates the migration of the germ plasm in a one-thousand cell blastula. At this stage the existent germ plasm has nearly all migrated to the four poles of the blastula whereas the mRNA elsewhere has nearly all degraded.

Since germ plasm migration and PGC development are reliant on functional maternal contribution (of certain mRNA) to the egg/zygote, alterations in the 3′-UTR of these mRNA will result in improper migration of PGC's and, ultimately, result in a reduction and/or incomplete absence of gametes in the adult fish that developed from the egg/zygote.

FIG. 2 shows further development of PGCs in fish embryos and in larva. As shown, a synthetic reporter mRNA encoding GFP and comprising an intact and functional 3′-UTR of a gene important to PCG migration. At 24 hours after fertilization (HAF), GFP signal is localized in discrete punctate and in later larva (at 48 HAF and 72 HAF), the GFP signal has traveled to gonadal ridge. Thus, 3′-UTR of a gene identified as important to PCG migration should be intact (i.e., not altered) to provide for proper PCG migration.

In methods of the present disclosure, the alteration in the 3′-UTR of the gene may results in a dysfunction in a maternally-expressed mRNA. As is known in the art, a maternally-expressed mRNA is deposited by a mother fish into her eggs to, at least, allow for rapid protein translation of the mRNA and, possibly, before the fertilized egg's transcriptions machinery has fully activated.

In the herein-disclosed methods, the maternally-expressed mRNA that comprises the dysfunction may be deposited into the egg by the female fish comprising the homozygous alteration. In embodiments, the dysfunction in the maternally-expressed mRNA prevents or reduces development and/or migration of PGCs in the fertilized egg, in a resulting zygote, and/or in a resulting larva.

The sterile fish may produce a reduced number of gametes relative to a fish resulting from fertilization of an egg obtained from a female fish lacking the homozygous alteration. In embodiments, the sterile fish fails to produce any gametes. The sterile fish may produce a reduced number of functional gametes relative to a fish resulting from fertilization of an egg obtained from a female fish lacking the homozygous alteration. In embodiments, the sterile fish fails to produce any functional gametes.

The fertilizing may be in vitro or the fertilizing may be in vivo and comprises mating a male fish and the female fish comprising the homozygous alteration. In the latter case, the mating may comprise internal fertilization of the egg or external fertilization of the egg.

In some cases, the method further comprises maintaining the fertilized egg, the resulting zygote, and/or the resulting larva under conditions suitable for development of the sterile fish into a fry the method further comprises maintaining the fry under conditions suitable for development of the sterile fish into a juvenile, and/or the method further comprises maintaining the juvenile under conditions suitable for development of the sterile fish into a fully grown, mature, and/or adult fish. Any known methods and steps for raising fish from fertilized egg to adulthood may be used with the present methods.

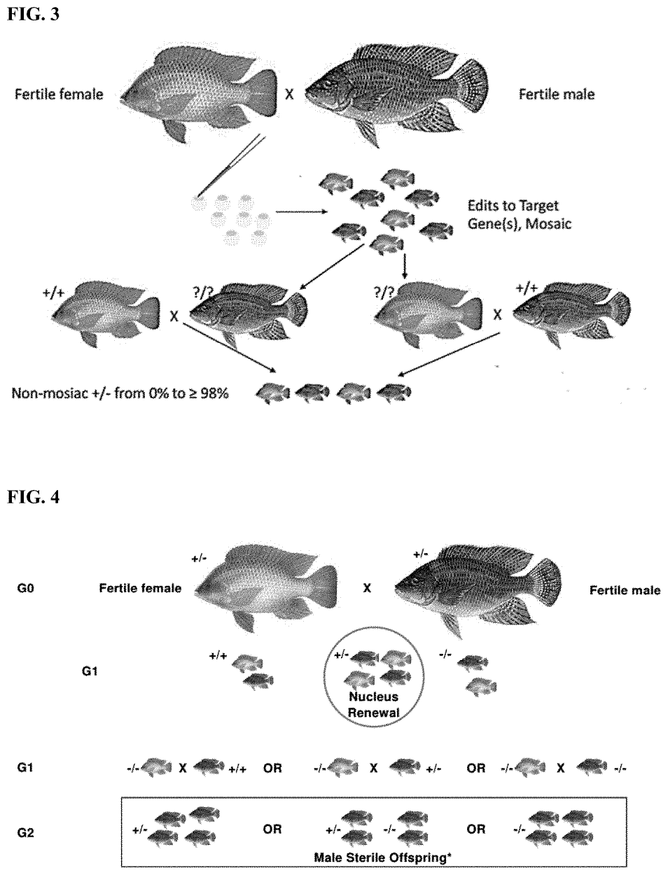

Moreover, standard breeding techniques can be used to create fish that are heterozygous homozygous for the alteration in the 3′-UTR of a gene and for generating sterile fish. See, e.g., FIG. 3 to FIG. 5.

Fish of any generation, including the sterile fish, may be genotyped to verify the presence of one or more alterations in the 3′-UTR of a gene. Standard laboratory methods for genotyping cells, tissues, and fish may be used. Alternately, the alteration may be identified by an expressed reporter/marker that indicates the presence of the alteration in the 3′-UTR of a gene. As described below, the alternation may be created by replacement of a native 3′-UTR with an exogenous gene, e.g., which encodes a reporter. In one example, the reporter is a fluorescent protein (e.g., GFP) which is expressed by cells/fish that carry the reporter. In this case, GFP may be used to verify the presence an alteration in the 3′-UTR of a gene.

In embodiments, the sterile fish is male. In embodiments, a substantial fraction of fertilized eggs from the female fish (that comprises a gene-edited homozygous alteration in the 3′-UTR of a gene) develop into sterile male fish. In embodiments, more than 50% of the fertilized eggs develop into sterile male fish. In embodiments, more than 60%, more than 75%, more than 80%, more than 85%, more than 90%, more than 95% of the fertilized eggs develop into sterile male fish. In embodiments, substantially all fertilized eggs from the female fish (that comprises a gene-edited homozygous alteration in the 3′-UTR of a gene) develop into sterile male fish.

The sperm used in fertilization may comprise an alteration in the 3′-UTR of the gene or the sperm may lack an alteration the 3′-UTR of the gene, i.e., may not have a gene-edited 3′-UTR.

In embodiments, the gene (when not gene-edited in its 3′-UTR) contributes to normal development and/or normal migration of primordial germ cells.

Numerous genes have been identified which are relevant to the development, maturation, and/or migration of primordial germ cells (PGCs). Non-limiting examples of these genes include nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, and puf/puf-A.

As examples, the dnd1/dnd gene encodes a protein that binds to microRNA-targeting sequences of mRNAs, thereby inhibiting microRNA-mediated repression. Reduced expression of this gene has been implicated in tongue squamous cell carcinoma. It is an RNA-binding factor that positively regulates gene expression by prohibiting miRNA-mediated gene suppression. It relieves miRNA repression in germline cells (by similarity) and prohibits the function of several miRNAs by blocking the accessibility of target mRNAs. It is believed to play a role during PGC survival but may not be essential for PGC migration.

The ddx4/vasa gene encodes an RNA binding protein with an ATP-dependent RNA helicase that is a member of the DEAD box family of proteins. The vasa gene is essential for germ cell development. The Vasa protein is found primarily in germ cells in embryos and adults throughout the animal kingdom, where it is involved in germ cell determination and function, as well as in multipotent stem cells, where its exact function is unknown.

The protein expressed by the nanos3/nanos/nanos1 gene plays a role in the maintenance of the undifferentiated state of germ cells regulating the spermatogonia cell cycle and inducing a prolonged transit in G1 phase. It effects cell proliferation, likely by repressing translation of specific mRNAs. It maintains the germ cell lineage by suppressing both Bax-dependent and -independent apoptotic pathways. It has been shown to be essential in the early stage embryo to protect the migrating PGCs from apoptosis.

The nucleotide sequences for the above-mentioned genes for which a 3′-UTR is altered are publicly available, for example at the NCBI databases. Based, in part, on the public information, identifying sites within a 3′-UTR that are suitable for alteration are well-within the ability of a skilled artisan. Using publicly available information, sequences alignments for several fish species has already identified deletion targets in their 3′-UTRs. Such conserved regions are likely required for proper function of the 3′-UTR. Moreover, should a gene for a specific species of fish not yet be available, the skilled artisan could identify 3′-UTR sequences using a standard homology analysis and by relying on a species for which the gene has been sequenced.

The alteration (in the 3′-UTR of the gene) may have been gene-edited in an unfertilized egg or in a fertilized egg. In embodiments, the egg that was gene-edited was obtained from a progenitor of the female fish comprising the homozygous alteration.

The alteration (in the 3′-UTR of the gene) may have been gene-edited in a zygote. In embodiments, the egg that was gene-edited was obtained from a progenitor of the female fish comprising the homozygous alteration.

In some cases, the method further comprises obtaining a cell's nucleus comprising the alteration in the 3′-UTR of the gene and further transferring the nucleus comprising the alteration to an enucleated egg; the enucleated egg receiving the nucleus develops into the progenitor of the female fish comprising the homozygous alteration.

Somatic cell nuclear transfer (SCNT) is a strategy for cloning a viable embryo from a body cell and an egg cell. As used herein the term “cloning” means production of genetically identical organisms asexually. SCNT comprises obtaining an enucleated egg and implanting a donor nucleus from a somatic (body) cell to produce a re-nucleated egg which develops, according to methods of the present disclosure, into the progenitor of the female fish comprising the homozygous alteration. Methods for performing SCNT are well known in the art.

Notably, in some cases, the alteration in the 3′-UTR of the gene may have been gene-edited in the cell providing the nucleus or was gene-edited in a parent cell. In the former case, a cell that provides the donor nucleus may be de novo gene-edited. In the latter case, cells may be grown and expanded in culture and their nuclei harvested and used in SCNT. Alternately, cells from a fish that comprises the alteration in the 3′-UTR of the gene may be used to provide donor nuclei. The donor fish may be homozygous for the alteration or may be heterozygous for the alteration.

The gene-editing steps may employ one or more of microinjection, lipid-based transfection, chemical-based transfection (e.g., calcium phosphate precipitation), electroporation, viral-mediated transduction, or exosome-mediated transfected, and a combination thereof. In embodiments, the micronuclear injection is pronuclear microinjection. In embodiments, the lipid-based transfection comprises nanoparticles, microparticles, or liposomes, and a combination thereof. Illustrative vectors for viral-mediated transduction include adenovirus, adeno-associated virus (AAV), lentivirus (e.g., modified HIV-1, SIV or FIV), and retrovirus (e.g., ASV, ALV or MoMLV).

Various techniques known in the art can be used introduce proteins and/or polynucleotides into cells (e.g., eggs) for gene-editing. Such techniques include, without limitation, pronuclear microinjection (U.S. Pat. No. 4,873,191), retrovirus mediated gene transfer into germ lines (Van der Putten et al., Proc. Natl. Acad. Sci. USA, 82:6148-6152, 1985), gene targeting into embryonic stem cells (Thompson et al., Cell, 56:313-321, 1989), electroporation of embryos (Lo, Mol. Cell. Biol., 3:1803-1814, 1983), sperm-mediated gene transfer (Lavitrano et al., Proc. Natl. Acad. Sci. USA, 99:14230-14235, 2002; Lavitrano et al., Reprod. Fert. Develop., 18:19-23, 2006), and in vitro transformation of somatic cells or stem cells, followed by nuclear transplantation (Wilmut et al., Nature, 385:810-813, 1997; and Wakayama et al., Nature, 394:369-374, 1998).

The initial gene-editing that produced the alteration in the 3′-UTR of the gene may have occurred in one, two, or more generations before the female fish (that comprises a gene-edited homozygous alteration in the 3′-UTR of a gene). In some cases, the progenitor precedes the female fish by at least one generation, at least two generations, at least three generations, at least five generations, at least ten generations, or at least one hundred generations, and any number of generations therebetween. In other words, once gene-editing has occurred in a progenitor and the alteration has been stably inherited, it may be unnecessary to perform further gene-editing, i.e., to the 3′-UTR of genes that are relevant to the development, maturation, and/or migration of primordial germ cells (PGCs).

Methods for creating gene-edited fish may follow the scheme shown in FIG. 3. Here, fertilized fish eggs from wild-type male and female fish are injected with a gene-editing cocktail to produce the desired genetic changes. These injected fish will be mosaic in the inheritance of any gene-edit changes, such that outcross of the injected fish will result in gene-edits from 0 to greater than or equal to 98%, depending on the efficiency of the gene-editing cocktail and delivery. In embodiments, the male and female fish that fertilize an egg may not be wild type. Instead, a fish line comprising an improved trait may be used for gene-editing the 3′-UTR of a gene relevant to PGC migration and development. Any fish line available and comprising an improved trait may be gene-edited according to methods of the present disclosure to produce sterile fish comprising the improved trait.

Because the proper migration of PGCs rely on the mothers' mRNA deposited into the egg, +/− mothers (with “+” being a wild-type 3′-UTR for a gene and “−” having an alteration in the 3′-UTR of the gene) may have offspring that are fertile −/− females and fertile −/− males. These fertile −/− females arose from a +/− mother, who deposited some maternal mRNAs that function in PGC migration and development, which allow the −/− females' PGCs to form into gametes. However, these fertile −/− females (who are unable to deposit functional maternal mRNAs) can only give rise to sterile offspring who will be phenotypically male in some fish species. Such offspring will have defects in PGC development, and therefore produce sterile male offspring with standard Mendelian inheritance of the targeted modification. Indeed, when −/− females are crossed with either the +/− males or the −/− males, the resulting offspring will be sterile males.

Males may also be produced by a +/− mother that are −/− and fertile. Mating of these −/− fertile males with −/− fertile females will always give rise to sterile males due to the homozygous alteration in the female

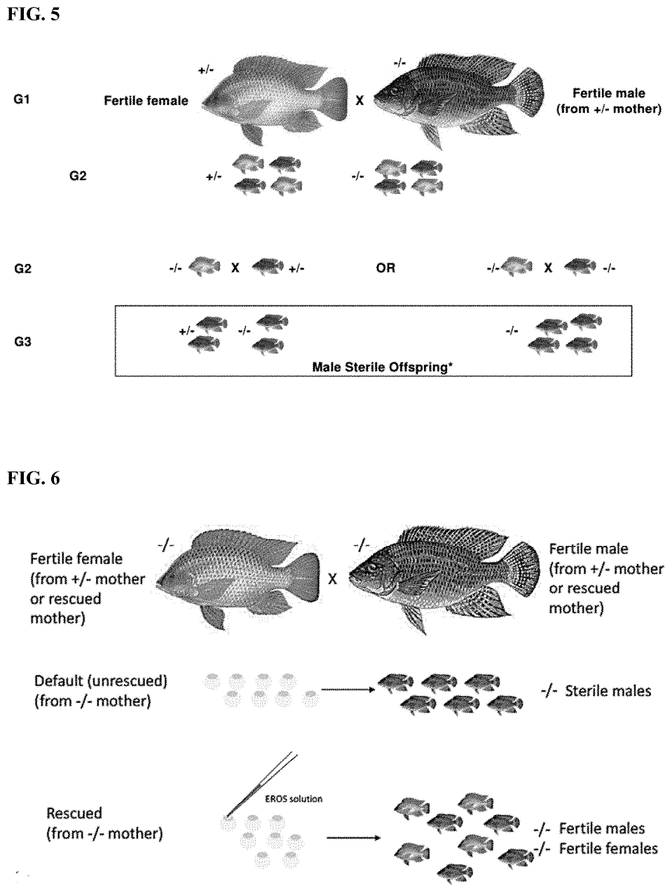

These steps are further shown in FIG. 4 and FIG. 5 and discussed later.

### Gene-Editing Tools

Gene editing tools have impacted the fields of biotechnology, gene therapy and functional genomic studies in many organisms by allowing targeted modification of DNA sequences, e.g., gene-editing.

In embodiments of the present disclosure, the gene-editing comprises use of a nuclease. The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Any nuclease known in the art that is capable of performing gene-editing may be used in methods of the present disclosure.

In some cases, the gene-editing comprises a site-specific gene editing system. The gene editing system may comprise CRISPR/CaS, a TALEN, a zinc finger nuclease, or a meganuclease.

More recently, RNA-guided endonucleases (RGENs) are directed to their target sites by a complementary RNA molecule. RGENs are examples of targeted nuclease systems: these system have a DNA-binding member that localizes the nuclease to a target site. The site is then cut by the nuclease. The Cas9/CRISPR system is a REGEN. CRISPRs (clustered regularly interspaced short palindromic repeats) comprises segments of prokaryotic DNA containing short repetitions of base sequences. Each repetition is followed by short segments of “spacer DNA” from previous exposures to a bacterial virus or plasmid. “Cas9” (CRISPR associated protein 9) is an RNA-guided DNA endonuclease enzyme associated with the CRISPR. By delivering the Cas9 protein and appropriate guide RNAs into a cell, the organism's genome can be cut at any desired location. Other Cas proteins are known in the art. tracrRNA is another RGEN tool.

TALENs and ZFNs have the nuclease fused to the DNA-binding member.

Transcription activator-like effector nucleases (TALENs) are another technology for gene editing are artificial restriction enzymes generated by fusing a TAL effector DNA-binding domain to a DNA cleavage domain. A DNA target site for binding a TALEN is determined and a fusion molecule comprising a nuclease and a series of Repeat Variable Diresidue (RVDs) that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. TALENs have been shown to induce gene modification in immortalized human cells by means of the two major eukaryotic DNA repair pathways, non-homologous end joining (NHEJ) and homology directed repair. TALENs may gene-edit with or without use of a separate polynucleotide, e.g., which acts as a Homology directed repair (HDR) template.

Zinc finger nucleases (ZFNs) are another technology useful for gene editing and are a class of engineered DNA-binding proteins that facilitate targeted editing of the genome by creating double-strand breaks in DNA at user-specified locations. ZFNs are artificial restriction enzymes generated by fusing a zinc finger DNA-binding domain to a DNA-cleavage domain. Zinc finger domains can be engineered to target desired DNA sequences, and this enables zinc-finger nucleases to target unique sequences within complex genomes. By taking advantage of endogenous DNA repair machinery, these reagents can be used to alter the genomes of higher organisms. ZFNs may be used in method of inactivating genes. Materials and methods for using zinc fingers and zinc finger nucleases for making gene-edited cells and organisms are disclosed in, e.g., U.S. Pat. No. 8,106,255; U.S. 2012/0192298; U.S. 2011/0023159; and U.S. 2011/0281306, the contents of each of which is incorporated by references in their entireties.

Meganuclease are another technology useful for gene editing in methods of the present disclosure. This system uses endodeoxyribonucleases characterized by a large recognition site (double-stranded DNA sequences of 12 to 40 base pairs); as a result, this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

The present methods may be adapted to use any gene editing system known now or in the future.

In embodiments, the gene editing system creates an alteration that comprises a deletion in the 3′-UTR, e.g., a deletion which results in a premature truncation of the 3′-UTR. In embodiments, the deletion prevents normal development and/or normal migration of primordial germ cells and/or reduces or abolishes recognition of the 3′-UTR by its binding protein. The gene editing system may create an alteration that comprises an insertion of a nucleic acid sequence into the 3′-UTR.

The gene-editing may comprise a polynucleotide.

In embodiments, the polynucleotide comprises one or more regions homologous to the gene's 3′-UTR nucleotide sequence and/or the polynucleotide may comprise one or more regions non-homologous to the gene's 3′-UTR nucleotide sequence. In embodiments, the polynucleotide comprises a homology directed repair (HDR) template or the polynucleotide comprises a guide RNA (gRNA). In embodiments, the gene-editing comprises a polynucleotide and a guide RNA (gRNA).

HDR is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations.

In embodiments, polynucleotide comprises a non-homologous region which may be a sequence that prevents normal development and/or normal migration of primordial germ cells. The sequence that prevents normal development and/or normal migration of primordial germ cells may reduce or abolish recognition of the 3′-UTR by its binding protein and/or may comprise one or more additional nucleotides.

In some cases, the sequence that prevents normal development and/or normal migration of primordial germ cells comprises a coding sequence for an exogenous gene.

The exogenous gene, via a gene-editing system, may replace an endogenous 3′-UTR. Here, the homologous regions may help the polynucleotide target the 3′-UTR and position the non-homologous sequences in the polynucleotide to replace a portion of the endogenous 3′-UTR.

In embodiments, the exogenous gene encodes a reporter, e.g., a fluorescent protein. The fluorescent protein may be a derivative or variant of green-fluorescent protein (GFP). Derivatives and variants of GFP are well known in the art.

The coding sequence for the exogenous gene may comprise a promoter, e.g., a constitutive promoter or a tissue-specific promoter. In embodiments, the sequences comprise polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like.

A polynucleotide useful in methods of the present disclosure may be a dsDNA or a single-stranded DNA (ssDNA). ssDNA templates may be from about 20 to about 5000 nucleotides in length, although other lengths can be used.

When serving as an HDR template, the polynucleotides may include random collections of nucleotides that abolish the 3′-UTR's binding site to binding protein. Or, it may comprise sequences that are generally similar to a 3′-UTR but lack one or more nucleotides necessary for recognition/binding by its binding protein. An HDR-functioning polynucleotide may be designed to replace a portion (up to a majority) of the 3′-UTR, thereby creating a truncated 3′-UTR.

The polynucleotide may comprise a sequence that is bound to a targeted nuclease system, and is thus the cognate binding site for the system's DNA-binding member. The term cognate refers to two biomolecules that typically interact, for example, a receptor and its ligand. In the context of gene-editing, one of the biomolecules may be designed with a sequence to bind with an intended, i.e., cognate, DNA site or protein site.

In some cases, the gene-editing occurs in an unfertilized egg, a fertilized egg, or a zygote having a genome that comprise an improved trait relative to a wild-type fish of similar species. The improved trait may be the result of genetic engineering and/or the result of selective breeding.

Methods for genetic engineering an improved trait may follow the scheme shown in FIG. 3. In embodiments, fertilized fish eggs from wild-type male and female fish are injected with a genetic-engineering cocktail to produce the desired genetic changes. These injected fish will be mosaic in the inheritance of any genetic changes, such that outcross of the injected fish will result in gene modification from 0 to greater than or equal to 98%, depending on the efficiency of the genetic engineering cocktail and delivery. In methods of the present disclosure, these steps may not be necessary; instead, a fish line comprising the improved trait may be used for gene-editing the 3′-UTR of a gene relevant to PGC migration and development. Any fish line available and comprising an improved trait may be gene-edited according to methods of the present disclosure to produce sterile fish comprising the improved trait.

The improved trait may any trait that has been introduced or bred into fish and that enhances the value of a commercial fish Examples include one or more of area of fat depot, body shape, disease resistance, faster growth, fat percentage, flesh color, greater protein content, improved fertility, larger muscles, skin color, and temperature tolerance. Accordingly, the methods of the present disclosure produce sterile fish that further comprise an improved trait; these improved traits would allow the fish to outcompete the wild populations of fish should the fish escape and were it fertile. Fortunately, the methods of the present disclosure produce sterile fish which are unable to transmit its improved genes into wild populations of fish and, possibly, reducing diversity in the wild.

### Gene-Edited Fish and Cells

As used herein, a fish is of the superclass Osteichthyes. The fish may be a ray-finned fish of the class Actinopterygii. In some cases, the fish is a bony fish of the infraclass Teleostei. In embodiments, a fish of the present disclosure is tilapia (e.g., Mozambique tilapia (Oreochromis mossambicus) and Nile tilapia (Oreochromis niloticus); salmon (e.g., Atlantic salmon (Salmo salar), Chinook salmon (Oncorhynchus tshawytscha), and Coho salmon (Oncorhynchus kisutch)); trout (e.g., Rainbow trout (Oncorhynchus mykiss*); tuna (e.g., Bluefin Tuna (Thunnus thynnus); seabass (e.g., European seabass (Dicentrarchus labrax); bream (e.g., White amur bream (Parabramis pekinensis); seabream (e.g., Red seabream* (Pagrus major*); barramundi* (Lates calcarifer); milkfish (Chanos chanos); catla (Catla catla*); carp (e.g., Crucian carp (Carassius carassius), Mud carp (Cirrhinus molitorella*), Mrigal carp (Cirrhinus mrigala), Grass carp (Ctenopharyngodon idellus), Common carp (Cyprinus carpio), Silver carp (Hypophthalmichthys molitrix), Bighead carp (Hypophthalmichthys nobilis*), Roho labeo (Labeo rohita), Black carp (Mylopharyngodon piceus)); catfish (e.g., Channel catfish (Ictalurus punctatus)); amberjack (e.g., Japanese amberjack (Seriola quinqueradiata); or zebrafish (Danio rerio).

Any fish species amenable to gene-editing may be used in the methods of the present disclosure. A skilled artisan would readily be able to adapt the techniques described herein with other species of bony fish.

Another aspect of the present disclosure is a fish comprising a heterozygous alteration in the three prime untranslated region (3′-UTR) of a gene.

Yet another aspect of the present disclosure is a fish comprising a homozygous alteration in the three prime untranslated region (3′-UTR) of a gene.

In these aspects, the gene contributes to normal development and/or normal migration of primordial germ cells. The gene may be nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, or puf/puf-A.

In some cases, the alteration in the 3′-UTR of the gene reduces or abolishes recognition of the 3′-UTR by its binding protein. In embodiments, the alteration comprises a deletion in the 3′-UTR, e.g., a premature truncation of the 3′-UTR. In embodiments, the deletion prevents normal development and/or normal migration of primordial germ cells. In embodiments, the alteration comprises an insertion of a nucleic acid sequence into the 3′-UTR, e.g., a nucleic acid sequence that comprises a coding sequence for an exogenous gene. The exogenous gene may encode a reporter. In embodiments, the insertion prevents normal development and/or normal migration of primordial germ cells.

The fish may be a female tilapia (e.g., Mozambique tilapia (Oreochromis mossambicus) and Nile tilapia (Oreochromis niloticus); salmon (e.g., Atlantic salmon (Salmo salar), Chinook salmon (Oncorhynchus tshawytscha), and Coho salmon (Oncorhynchus kisutch)); trout (e.g., Rainbow trout (Oncorhynchus mykiss*); tuna (e.g., Bluefin Tuna (Thunnus thynnus); seabass (e.g., European seabass (Dicentrarchus labrax); bream (e.g., White amur bream (Parabramis pekinensis); seabream (e.g., Red seabream* (Pagrus major*); barramundi* (Lates calcarifer); milkfish (Chanos chanos); catla (Catla catla*); carp (e.g., Crucian carp (Carassius carassius), Mud carp (Cirrhinus molitorella*), Mrigal carp (Cirrhinus mrigala), Grass carp (Ctenopharyngodon idellus), Common carp (Cyprinus carpio), Silver carp (Hypophthalmichthys molitrix), Bighead carp (Hypophthalmichthys nobilis*), Roho labeo (Labeo rohita), Black carp (Mylopharyngodon piceus)); catfish (e.g., Channel catfish (Ictalurus punctatus)); amberjack (e.g., Japanese amberjack (Seriola quinqueradiata); or zebrafish (Danio rerio).

In some cases, the fish further comprises an improved trait relative to a wild-type fish of similar species. The improved trait may be the result of genetic engineering and/or the improved trait is the result of selective breeding. The improved trait may any trait that has been introduced or bred into fish and that enhances the value of a commercial fish. The improved trait may be one or more of area of fat depot, body shape, disease resistance, faster growth, fat percentage, flesh color, greater protein content, improved fertility, larger muscles, skin color, and temperature tolerance.

In an aspect, the present disclosure provides a sterile fish obtained by any herein-disclosed method.

In another aspect, the present disclosure provides a food product comprising tissue obtained from the sterile fish obtained by any herein-disclosed method.

An aspect of the present disclosure is an in vitro cell. The in vitro cell comprises a heterozygous alteration in the three prime untranslated region (3′-UTR) of a gene or a homozygous alteration in the 3′-UTR of a gene. In these aspects, the gene contributes to normal development and/or normal migration of primordial germ cells.

In embodiments, the in vitro cell is a somatic cell, an unfertilized egg, a fertilized egg, or a sperm cell.

Another aspect of the present disclosure is an in vivo cell. The in vivo cell comprises a heterozygous alteration in the three prime untranslated region (3′-UTR) of a gene or a homozygous alteration in the 3′-UTR of a gene. In these aspects, the gene contributes to normal development and/or normal migration of primordial germ cells.

In embodiments, the in vivo cell is a somatic cell, an unfertilized egg, a fertilized egg, or a sperm cell.

### Embryonic Rescue of Sterility

An aspect of the present disclosure is a method to rescue the sterility in a fish resulting from a gene-edited alteration in the three prime untranslated region (3′-UTR) of a gene responsible for germ plasm migration and/or gamete development. The method comprises injecting into an egg from the fish a wild type copy of mRNA corresponding to the gene that comprises gene-edited alteration.

In embodiments, the offspring of the egg would be fertile but the offspring produces sterile offspring.

This aspect is referred to as Embryonic Rescue of Sterility. This involves rescuing embryos that are homozygous for the alteration (in the 3′-UTR) and which would be sterile when matured. Here, embryos are treated with wild-type mRNA of the gene-edited (which has the 3′-UTR alteration). This treatment will allow for successful specification and migration of primordial germ cells. This results in functional gonadal development and fertile fish. See, FIG. 6.

### Definitions

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

Any aspect or embodiment described herein can be combined with any other aspect or embodiment as disclosed herein.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Genetically Engineering Fish

FIG. 3 illustrates one embodiment of making genetically engineered (including gene-edited) fish. The images in FIG. 3 exemplify the fish as a tilapia, but any fish, e.g., bony fish, may be used in the below-described methods. In the case of gene-editing, a gene edit cocktail comprises of a specific nuclease are used to target specific gene targets in fertilized fish eggs. The fish eggs may be wild-type or comprise a improved trait (e.g., area of fat depot, body shape, disease resistance, faster growth, fat percentage, flesh color, greater protein content, improved fertility, larger muscles, skin color, and temperature tolerance). Examples of nuclease include zinc finger nuclease (ZFN); Clustered Randomly Spaced Palindromic Repeats (CRISPR); Transcription activator-like effector nucleases (TALENs); Meganucleases or the like. The cocktail can be introduced into the embryo in any of the ways known to the art. For example, the cocktail may be introduced by microinjection, transfection, electroporation, lipofection or the like. The fish modified by the cocktail will be mosaic for the inheritance of any gene edits. It should be appreciated that the chimeric fish resulting from gene modification may be homozygous or heterozygous for the modification in a spectrum of cells ranging from almost complete modification in the cells to a limited number of modified cells. Oocytes from injected mothers inherit a single copy of a gene that has been targeted for modification, either wild-type or modified. The efficiency of that inheritance reflects the overall efficiency of the gene edit. Of course, those of skill in the art will appreciate that the modified offspring will develop into both males and females. Thus, there will be a population of fish arising from the genetic edit of the 3′-UTR that are heterozygous (+/−), they are generation G0 (in FIG. 4). Mating of these G0 fish will result in a first-generation offspring (G1) having Mendelian inheritance patterns for the modification (+/+, +/− and −/−) in which +/+ will have a wild type phenotype, +/− while will have a wild type phenotype but which are heterozygous and a cohort having −/− phenotype. Here, “+” being a wild-type 3′-UTR for a gene and “−” having an alteration in the 3′-UTR of the gene.

Those of skill will appreciate that some fish, which develop as sterile will be monosex. Monosex refers to the culture of either all-male or all-female populations, a sought-after approach in commercial fish farming. For example, tilapia and zebrafish will develop as male when gametogenesis fails (e.g., sterile), salmon on the other hand appear to develop as a mix of males and females with incomplete gamete development.

Because the proper migration of PGCs rely on the mothers' mRNA deposited with the egg, +/− mothers may have offspring that are −/− females and −/− male that are fertile. These fertile −/− females can only give rise to sterile offspring who will be phenotypically male in some fish species. Similarly, males may be produced by a +/− mother that are −/− and fertile. Mating of these −/− fertile males with −/− fertile females will always give rise to sterile males (in the case of Tilapia), as shown in FIG. 4 and FIG. 5, and discussed later.

It will also be appreciated that the scheme shown in FIG. 3 can be used to introduce improved traits, as described herein, into fertilized eggs.

### Example 2: Illustrative Sterile Male Breeding Programs

As shown in FIG. 4, fish heterozygous (+/−) for alterations in the 3′-UTR of a gene (relevant to PGC migration and development). The heterozygous fish are crossed and the resulting offspring, G1, will contain a normal mix of males and females with typical Mendelian inheritance of the alteration. The heterozygous G1 fish can be selected for growth and breeding characteristics to improve the desired fish and/or may be used to renew the breeding nucleus as new G0 parents (“nucleus renewal”).

The G1 generation will include fertile females having homozygous alterations in the 3′-UTR; these females arose from a +/− mother, who deposited some maternal mRNAs that function in PGC migration and development, which allow the −/− females' PGCs to form into gametes.

When females having homozygous alterations in the 3′-UTR of the gene are mated to any male, the resulting G2 offspring will lack any functional maternal-contributed mRNA for the gene (relevant to PGC migration and development). Such offspring will have defects in PGC development, and therefore produce sterile male offspring with standard Mendelian inheritance of the targeted modification.

### Example 3: Illustrative Female Expansion Breeding Programs

FIG. 5 illustrates how the numbers of gene-edited fertile −/− females can be amplified to facilitate production of increased numbers of sterile male offspring. This is accomplished by crossing a heterozygous fertile G1 female to a homozygous G1 fertile male, which must be obtained from a heterozygous mother. All the G2 progeny will be fertile: both +/− and −/− males and females. Normal Mendelian inheritance will result in expected male/female production rates with half of these being homozygous mutants. When the −/− females are crossed with either the +/− males or the −/− males, the resulting offspring will be sterile males.

Since breeding homozygous −/− females to any male will result in male sterile offspring regardless of the genotype of the male, amplifying the number of females homozygous for the alteration would produce a greater yield of sterile males in a population of farmed fish.

### Example 4: Illustrative Steps in Embryonic Rescue of Sterility

In cases where the homozygous mutants need to be propagated, sterility can be rescued in sterile animals by introduction of rescue cocktail comprising temporary instruction for the production of primordial germ cell in the sterile fish (FIG. 6). The rescue cocktail comprises mRNAs comprising wild-type 3′-UTRs for the altered gene and will provide temporary instruction for the production of primordial germ cells in the fish that developed from the injected eggs.

The result is proper migration of the germ plasm and development of correctly sexed gonads providing progeny of both sexes, both of which are fertile for a single generation. However, as the “rescued” fish are genetically −/− any subsequent, un-rescued progeny produced would also be −/− and would be phenotypically male and sterile. Therefore, injected fish would produce a generation of fertile male/female mixed offspring that are homozygous for the edit and whose offspring will also be homozygous for the mutation and sterile. As such, these rescuing steps would be necessary to continue to propagate a sterile line.

### Example 5: Methods for Producing Sterile Fish Via Gene-Editing

There are key elements within the 3′-UTR of germplasm mRNAs that regulate the integrity, translational potential, and localization of these mRNAs and their eventual gene products. These germplasm mRNAs are deposited by a mother into fish egg where they direct the development and migration of primordial germ cells (PGCs). Without proper germ cell development and migration, functional gonads will not be formed and will make the offspring sterile. Offspring from mothers that carry homozygous disruptions of key elements within the 3′-UTR of one or more germplasm mRNA should be sterile due to loss of proper PGC development or migration.

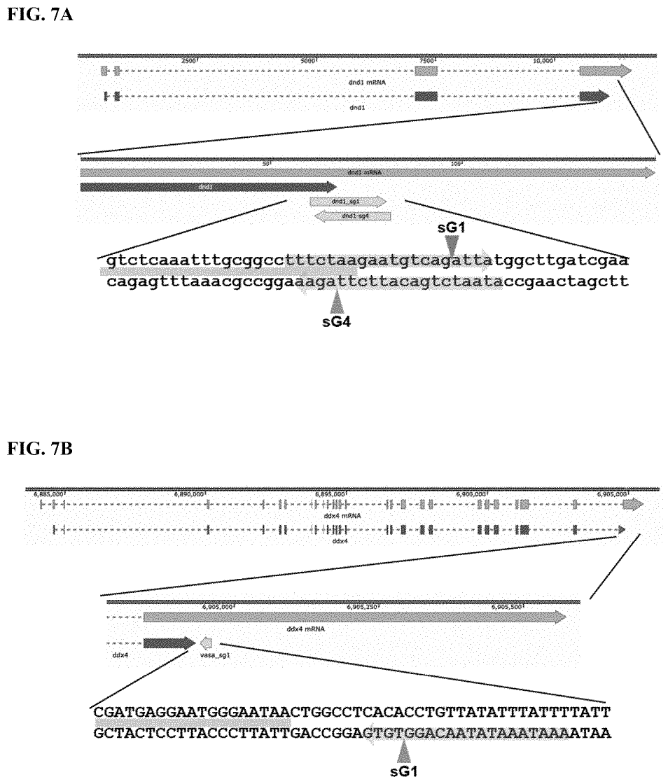

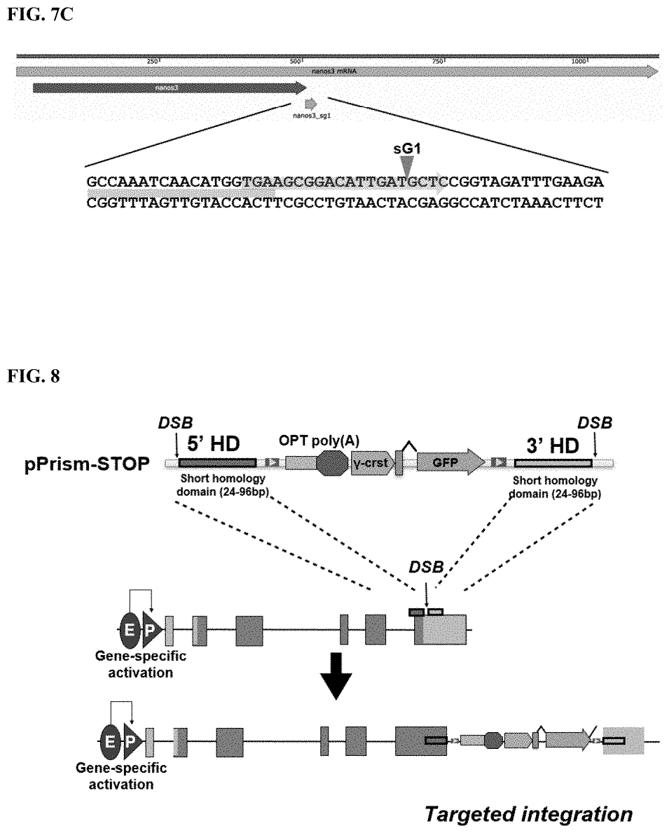

Here, zebrafish (Danio rerio) was used as a convenient fish model. The 3′-UTR of three genes relevant to PGC development or migration were characterized and targets for guide RNA (gRNA) were selected. See, FIG. 7A to FIG. 7C.

A targeting construct that targets and replaces (i.e., alters) the wild-type 3′-UTR of these genes was designed and constructed. See, for example FIG. 8. A targeting construct comprises homologous regions that allow specific binding to a target 3′-UTR and one or more non-homologous regions comprising a sequence that prevents normal development and/or normal migration of primordial germ cells. In the targeting construct of FIG. 8, the non-homologous region comprised a coding sequence for an exogenous gene (i.e., GFP). Thus, GFP could be used to mark and track cells comprising the altered 3′-UTR.

In other embodiments, a targeting construct lacked the coding sequence for an exogenous gene and merely disrupted the function of the 3′-UTR. In these embodiments, fish eggs were transfected with a targeting construct and with a marker construct that expresses GFP in PGCs. In embodiments, offspring of the fish that were transfected with the targeting construct were transfected with a marker construct that expresses GFP in PGCs. An example of the marker construct is shown at the top of FIG. 2.

Using a gene-editing system (here, CRISPER/Cas9; yet any other gene-editing system described herein would work), through targeted integration, the endogenous wild-type 3′-UTRs of three illustrative target genes (nanos3, dnd1, and vasa) was replaced with a targeting construct that altered the 3′-UTR such that it was non-functional.

For all three illustrative genes (nanos3, dnd1, and vasa), target integration and 3′-UTR replacement occurred precisely.

Germline founders were identified. F1 adults (first generation non-mosaic carriers) were identified. For dnd1 and vasa, F2 generations were obtained by outcross of F1 carriers to wild-type fish to propagate the lines. In addition, for both dnd1 and vasa, F2 offspring were produced by sibling crosses producing homozygous embryos for alterations in the dnd1 and vasa 3′-UTRs, respectively.

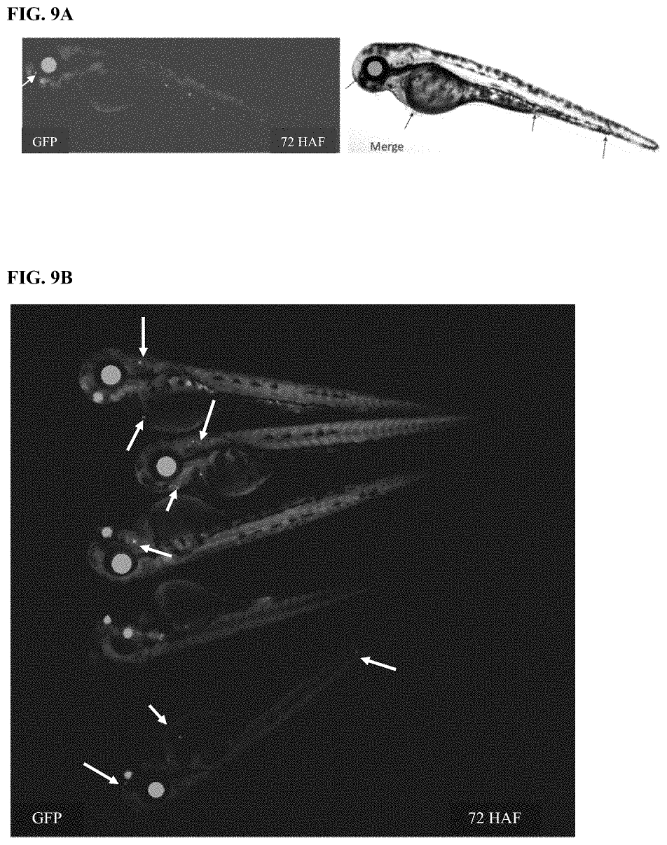

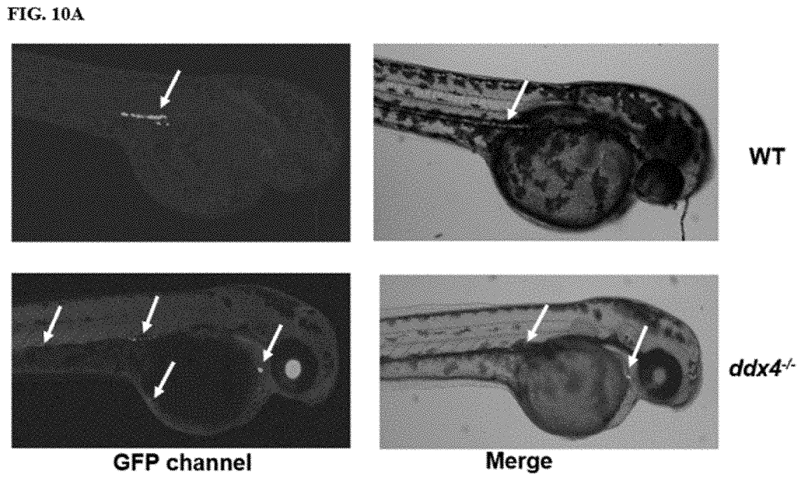

As shown in FIG. 9A and FIG. 9B, when injected with the GFP nanos3′-UTR marker construct mRNA, some offspring of the dnd1^{+/g1STOP} in-cross fish had PGCs that did not migrate normally. However, some PGSs still traveled to gonadal ridge; these properly migrated PGCs may be non-functional and not give rise to gametes. See the top fish in FIG. 9B. Compare to FIG. 2.

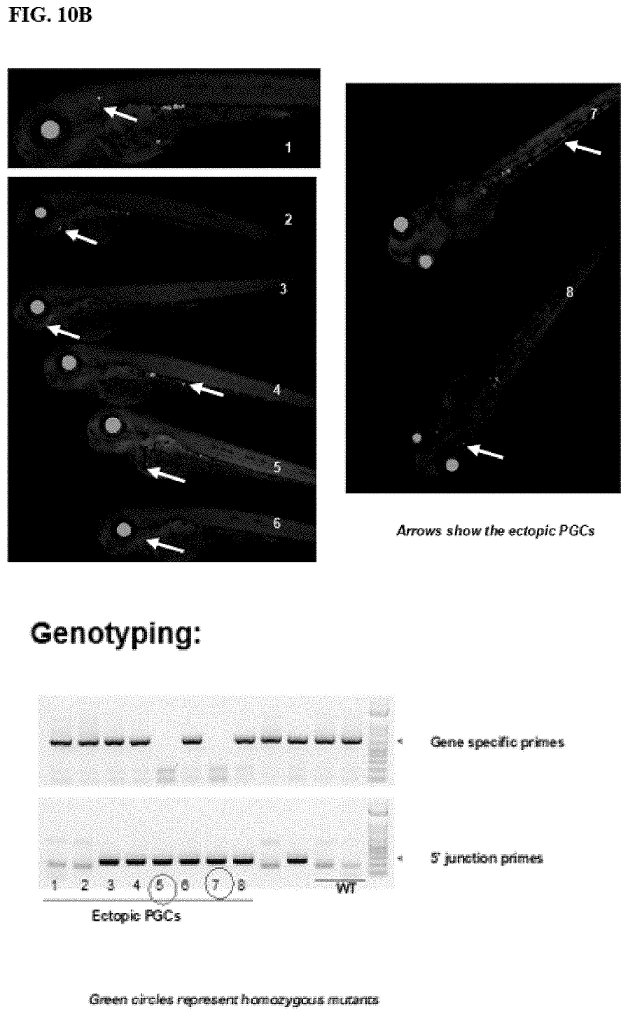

As shown in FIG. 10A and FIG. 10B, when injected with the GFP nanos3′-UTR marker construct mRNA, PGCs in ddx4/vasa^{-gRNA1} F2 embryos from a het in-cross of F1 parents had ectopic migration. Gel images to the right of FIG. 10B show genotyping of the fish.

Sterility of the resulting male fish are tested by crossing to fertile females.

Of course, in this example, the 3′-UTR of three genes were altered. Based on the teachings of the present disclosure and information skilled in the art, other genes relevant to PGC migration and development can be similarly altered. Moreover, in this example, zebrafish were used as a model fish. Based on the teachings of the present disclosure and information skilled in the art, one would understand that any bony fish amenable to gene-editing may be used.

Thus, a skilled artisan would readily be able to adapt the techniques described herein with another species of bony fish and for altering any relevant gene to PGC migration and/or development.

### Embodiments

The following paragraphs provide for various embodiments of the present invention.
Embodiment 1. A method of producing a sterile fish, the method comprising:
   fertilizing an egg with a sperm, wherein the egg is obtained from a female fish comprising a gene-edited homozygous alteration in the three prime untranslated region (3′-UTR) of a gene.
Embodiment 2. The method of embodiment 1, wherein the alteration in the 3′-UTR of the gene results in a dysfunction in a maternally-expressed mRNA.
Embodiment 3. The method of embodiment 1 or embodiment 2, wherein the maternally-expressed mRNA comprising the dysfunction is deposited into the egg by the female fish comprising the homozygous alteration.
Embodiment 4. The method of embodiment 2 or embodiment 3, wherein the dysfunction in the maternally-expressed mRNA prevents or reduces development and/or migration of primordial germ cells in the fertilized egg, in a resulting zygote, and/or in a resulting larva.
Embodiment 5. The method of any one of embodiments 1 to 4, wherein the sterile fish produces a reduced number of gametes relative to a fish resulting from fertilization of an egg obtained from a female fish lacking the homozygous alteration.
Embodiment 6. The method of any one of embodiments 1 to 5, wherein the sterile fish produces a reduced number of functional gametes relative to a fish resulting from fertilization of an egg obtained from a female fish lacking the homozygous alteration.
Embodiment 7. The method of any one of embodiments 1 to 6, wherein the fertilizing is in vitro.
Embodiment 8. The method of any one of embodiments 1 to 6, wherein the fertilizing is in vivo and comprises mating a male fish and the female fish comprising the homozygous alteration.
Embodiment 9. The method of any one of embodiments 1 to 8, further comprising maintaining the fertilized egg, the resulting zygote, and/or the resulting larva under conditions suitable for development of the sterile fish into a fry.
Embodiment 10. The method of embodiment 9, further comprising maintaining the fry under conditions suitable for development of the sterile fish into a juvenile.
Embodiment 11. The method of embodiment 10, further comprising maintaining the juvenile under conditions suitable for development of the sterile fish into a fully grown, mature, and/or adult fish.
Embodiment 12. The method of any one of embodiments 1 to 11, wherein the sterile fish is male.
Embodiment 13. The method of any one of embodiments 1 to 12, wherein the sperm comprises an alteration in the 3′-UTR of the gene or the sperm lacks an alteration the 3′-UTR of the gene.
Embodiment 14. The method of any one of embodiments 1 to 13, wherein the gene contributes to normal development and/or normal migration of primordial germ cells.
Embodiment 15. The method of any one of embodiments 1 to 14, wherein the gene is selected from group consisting of nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, and puf/puf-A.
Embodiment 16. The method of any one of embodiments 1 to 15, wherein the alteration was gene-edited in an unfertilized egg or in a fertilized egg, wherein the egg is obtained from a progenitor of the female fish comprising the homozygous alteration.
Embodiment 17. The method of any one of embodiments 1 to 15, wherein the alteration was gene-edited in a zygote resulting from fertilization of an egg obtained from a progenitor of the female fish comprising the homozygous alteration.
Embodiment 18. The method of any one of embodiments 1 to 17, further comprising obtaining a cell's nucleus comprising the alteration.
Embodiment 19. The method of embodiment 18, further comprising transferring the nucleus comprising the alteration to an enucleated egg, wherein the enucleated egg receiving the nucleus develops into the progenitor of the female fish comprising the homozygous alteration.
Embodiment 20. The method of embodiment 18 or embodiment 19, wherein the alteration was gene-edited in the cell providing the nucleus or was gene-edited in a parent cell.
Embodiment 21. The method of any one of embodiments 1 to 20, wherein the gene-editing comprises microinjection, lipid-based transfection, chemical-based transfection, electroporation, viral-mediated transduction, or exosome-mediated transfected, and a combination thereof
Embodiment 22. The method of embodiment 21, wherein the micronuclear injection is pronuclear microinjection.
Embodiment 23. The method of embodiment 21, wherein the lipid-based transfection comprises nanoparticles, microparticles, or liposomes, and a combination thereof.
Embodiment 24. The method of any one of embodiments 1 to 23, wherein the progenitor precedes the female fish by at least one generation, at least two generations, at least three generations, at least five generations, at least ten generations, or at least one hundred generations.
Embodiment 25. The method of any one of embodiments 1 to 24, wherein the gene-editing comprises use of a nuclease.
Embodiment 26. The method of any one of embodiments 1 to 25, wherein the gene-editing comprises a site-specific gene editing system.
Embodiment 27. The method of embodiment 26, wherein the gene editing system comprises CRISPR/CaS, a TALEN, a zinc finger nuclease, or a meganuclease.
Embodiment 28. The method of embodiment 26 or embodiment 27, wherein the gene editing system creates an alteration that comprises a deletion in the 3′-UTR.
Embodiment 29. The method of embodiment 28, wherein the deletion comprises a premature truncation of the 3′-UTR.
Embodiment 30. The method of embodiment 28 or embodiment 29, wherein the deletion prevents normal development and/or normal migration of primordial germ cells.
Embodiment 31. The method of any one of embodiments 28 to 30, wherein the deletion reduces or abolishes recognition of the 3′-UTR by its binding protein.
Embodiment 32. The method of any one of embodiments 26 to 31, wherein the gene editing system creates an alteration that comprises an insertion of a nucleic acid sequence into the 3′-UTR.
Embodiment 33. The method of any one of embodiments 1 to 32, wherein the gene-editing comprises a polynucleotide.
Embodiment 34. The method of embodiment 33, wherein the polynucleotide comprises one or more regions homologous to the gene's 3′-UTR nucleotide sequence.
Embodiment 35. The method of embodiment 33 or embodiment 34, wherein the polynucleotide comprises one or more regions non-homologous to the gene's 3′-UTR nucleotide sequence.
Embodiment 36. The method of any one of embodiments 33 to 35, wherein the polynucleotide comprises a homology directed repair (HDR) template.
Embodiment 37. The method of any one of embodiments 33 to 36, wherein the polynucleotide comprises a guide RNA (gRNA).
Embodiment 38. The method of any one of embodiments 33 to 36, wherein the gene-editing comprises a polynucleotide and a guide RNA (gRNA).
Embodiment 39. The method of any one of embodiments 35 to 38, wherein a non-homologous region comprises a sequence that prevents normal development and/or normal migration of primordial germ cells.
Embodiment 40. The method of embodiment 39, wherein the sequence that prevents normal development and/or normal migration of primordial germ cells reduces or abolishes recognition of the 3′-UTR by its binding protein.
Embodiment 41. The method of embodiment 39 or embodiment 40, wherein the sequence that prevents normal development and/or normal migration of primordial germ cells comprises one or more additional nucleotides.
Embodiment 42. The method of any one of embodiments 39 to 40, wherein the sequence that prevents normal development and/or normal migration of primordial germ cells comprises a coding sequence for an exogenous gene.
Embodiment 43. The method of embodiment 42, wherein the coding sequence for the exogenous gene comprises a promoter.
Embodiment 44. The method of embodiment 43, wherein the promoter is a constitutive promoter or is a tissue-specific promoter.
Embodiment 45. The method of any one of embodiments 42 to 44, wherein the exogenous gene encodes a reporter.
Embodiment 46. The method of embodiment 45, wherein the reporter is a fluorescent protein.
Embodiment 47. The method of embodiment 46, wherein the fluorescent protein is a derivative or variant of green-fluorescent protein (GFP).
Embodiment 48. The method of any one of embodiments 1 to 47, wherein the sterile fish is selected from tilapia (e.g., Mozambique tilapia (Oreochromis mossambicus) and Nile tilapia (Oreochromis niloticus); salmon (e.g., Atlantic salmon (Salmo salar), Chinook salmon (Oncorhynchus tshawytscha), and Coho salmon (Oncorhynchus kisutch)); trout (e.g., Rainbow trout (Oncorhynchus mykiss*); tuna (e.g., Bluefin Tuna (Thunnus thynnus); seabass (e.g., European seabass (Dicentrarchus labrax); bream (e.g., White amur bream (Parabramis pekinensis); seabream (e.g., Red seabream* (Pagrus major*); barramundi* (Lates calcarifer); milkfish (Chanos chanos); catla (Catla catla*); carp (e.g., Crucian carp (Carassius carassius), Mud carp (Cirrhinus molitorella*), Mrigal carp (Cirrhinus mrigala), Grass carp (Ctenopharyngodon idellus), Common carp (Cyprinus carpio), Silver carp (Hypophthalmichthys molitrix), Bighead carp (Hypophthalmichthys nobilis*), Roho labeo (Labeo rohita), Black carp (Mylopharyngodon piceus)); catfish (e.g., Channel catfish (Ictalurus punctatus)); amberjack (e.g., Japanese amberjack (Seriola quinqueradiata); and zebrafish (Danio rerio).
Embodiment 49. The method of any one of embodiments 1 to 48, wherein the female fish comprising a gene-edited homozygous alteration further comprises an improved trait relative to a wild-type fish of similar species.
Embodiment 50. The method of any one of embodiments 16 to 49, wherein the progenitor comprises an improved trait relative to a wild-type fish of similar species.
Embodiment 51. The method of embodiment 49 or embodiment 50, wherein the improved trait is the result of genetic engineering.
Embodiment 52. The method of any one of embodiments 49 to 51, wherein the improved trait is the result of selective breeding.
Embodiment 53. The method of any one of embodiments 49 to 52, wherein the improved trait is one or more of area of fat depot, body shape, disease resistance, faster growth, fat percentage, flesh color, greater protein content, improved fertility, larger muscles, skin color, and temperature tolerance.
Embodiment 54. A sterile fish obtained by the method of any one of embodiments 1 to 53.
Embodiment 55. A food product comprising tissue obtained from the sterile fish of embodiment 54.
Embodiment 56. A method to rescue the sterility in a fish resulting from a gene-edited alteration in the three prime untranslated region (3′-UTR) of a gene responsible for germ plasm migration and/or gamete development, the method comprising: injecting into an egg from the fish a wild type copy of mRNA corresponding to the gene that comprises gene-edited alteration.
Embodiment 57. The method of embodiment 56, wherein the offspring of the egg would be fertile but the offspring produces sterile offspring.
Embodiment 58. An in vitro cell comprising a heterozygous alteration in the three prime untranslated region (3′-UTR) of a gene, wherein the gene contributes to normal development and/or normal migration of primordial germ cells.
Embodiment 59. An in vitro cell comprising a homozygous alteration in the three prime untranslated region (3′-UTR) of a gene, wherein the gene contributes to normal development and/or normal migration of primordial germ cells.
Embodiment 60. The in vitro cell of embodiment 58 or embodiment 59, wherein the cell is a somatic cell, an unfertilized egg, a fertilized egg, or a sperm cell.
Embodiment 61. An in vivo cell comprising a heterozygous alteration in the three prime untranslated region (3′-UTR) of a gene, wherein the gene contributes to normal development and/or normal migration of primordial germ cells.
Embodiment 62. An in vivo cell comprising a homozygous alteration in the three prime untranslated region (3′-UTR) of a gene, wherein the gene contributes to normal development and/or normal migration of primordial germ cells.
Embodiment 64. The in vivo cell of embodiment 61 or embodiment 62, wherein the cell is a somatic cell, an unfertilized egg, or a sperm cell.
Embodiment 65. A fish comprising a heterozygous alteration in the three prime untranslated region (3′-UTR) of a gene, wherein the gene contributes to normal development and/or normal migration of primordial germ cells.
Embodiment 66. A fish comprising a homozygous alteration in the three prime untranslated region (3′-UTR) of a gene, wherein the gene contributes to normal development and/or normal migration of primordial germ cells.
Embodiment 67. The fish of embodiment 65 or embodiment 66, wherein the gene is selected from group consisting of nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, and puf/puf-A.
Embodiment 68. The fish of any one of embodiments 65 to 67, wherein the alteration reduces or abolishes recognition of the 3′-UTR by its binding protein.
Embodiment 69. The fish of any one of embodiments 65 to 68, wherein the alteration comprises a deletion in the 3′-UTR.
Embodiment 70. The fish of embodiment 69, wherein the deletion comprises a premature truncation of the 3′-UTR.
Embodiment 71. The fish of embodiment 69 or embodiment 70, wherein the deletion prevents normal development and/or normal migration of primordial germ cells.
Embodiment 72. The fish of any one of embodiments 65 to 68, wherein the alteration comprises an insertion of a nucleic acid sequence into the 3′-UTR.
Embodiment 73. The fish of embodiment 72, wherein the nucleic acid sequence comprises a coding sequence for an exogenous gene.
Embodiment 74. The fish of embodiment 73, wherein the exogenous gene encodes a reporter.
Embodiment 75. The fish of any one of 72 to 74, wherein the insertion prevents normal development and/or normal migration of primordial germ cells.
Embodiment 76. The fish of any one of embodiments 65 to 75, wherein the fish is selected from tilapia (e.g., Mozambique tilapia (Oreochromis mossambicus) and Nile tilapia (Oreochromis niloticus); salmon (e.g., Atlantic salmon (Salmo salar), Chinook salmon (Oncorhynchus tshawytscha), and Coho salmon (Oncorhynchus kisutch)); trout (e.g., Rainbow trout (Oncorhynchus mykiss*); tuna (e.g., Bluefin Tuna (Thunnus thynnus); seabass (e.g., European seabass (Dicentrarchus labrax); bream (e.g., White amur bream (Parabramis pekinensis); seabream (e.g., Red seabream* (Pagrus major*); barramundi* (Lates calcarifer); milkfish (Chanos chanos); catla (Catla catla*); carp (e.g., Crucian carp (Carassius carassius), Mud carp (Cirrhinus molitorella*), Mrigal carp (Cirrhinus mrigala), Grass carp (Ctenopharyngodon idellus), Common carp (Cyprinus carpio), Silver carp (Hypophthalmichthys molitrix), Bighead carp (Hypophthalmichthys nobilis*), Roho labeo (Labeo rohita), Black carp (Mylopharyngodon piceus)); catfish (e.g., Channel catfish (Ictalurus punctatus)); amberjack (e.g., Japanese amberjack (Seriola quinqueradiata); and zebrafish (Danio rerio).
Embodiment 77. The fish of any one of embodiments 65 to 76, wherein the fish is female.
Embodiment 78. The fish of embodiment 77, wherein the female fish further comprises an improved trait relative to a wild-type fish of similar species.
Embodiment 79. The fish of embodiment 78, wherein the improved trait is the result of genetic engineering.
Embodiment 80. The fish of embodiment 78 or embodiment 79, wherein the improved trait is the result of selective breeding.
Embodiment 81. The fish of any one of embodiments 78 to 80, wherein the improved trait is one or more of area of fat depot, body shape, disease resistance, faster growth, fat percentage, flesh color, greater protein content, improved fertility, larger muscles, skin color, and temperature tolerance.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of producing a sterile fish, the method comprising:
fertilizing an egg with a sperm, wherein the egg is obtained from a female fish comprising a gene-edited homozygous alteration in the three prime untranslated region (3′-UTR) of a gene.

2. The method of claim 1, wherein the alteration in the 3′-UTR of the gene results in a dysfunction in a maternally-expressed mRNA that is deposited into the egg by the female fish comprising the homozygous alteration, wherein the dysfunction in the maternally-expressed mRNA prevents or reduces development and/or migration of primordial germ cells in the fertilized egg, in a resulting zygote, and/or in a resulting larva.

3. The method of claim 1, wherein the sterile fish produces a reduced number of gametes relative to a fish resulting from fertilization of an egg obtained from a female fish lacking the homozygous alteration.

4. The method claim 1, wherein the fertilizing is in vitro or in vivo, wherein in vivo comprises mating a male fish and the female fish comprising the homozygous alteration.

5. The method of claim 1, further comprising maintaining the fertilized egg, the resulting zygote, and/or the resulting larva under conditions suitable for development of the sterile fish into a fry, further comprising maintaining the fry under conditions suitable for development of the sterile fish into a juvenile, and/or further comprising maintaining the juvenile under conditions suitable for development of the sterile fish into a fully grown, mature, and/or adult fish.

6. The method of any one of claim 1, wherein the sterile fish is male.

7. The method of claim 1, wherein the gene contributes to normal development and/or normal migration of primordial germ cells and is selected from group consisting of nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, and puf/puf-A.

8. The method of claim 1, wherein the alteration was gene-edited in a fertilized egg or in an unfertilized egg, wherein the egg is obtained from a progenitor of the female fish comprising the homozygous alteration or was gene-edited in a zygote resulting from fertilization of an egg obtained from the progenitor of the female fish comprising the homozygous alteration.

9. The method claim 1, wherein the method comprises obtaining a cell's nucleus from a cell comprising the alteration and transferring the nucleus to an enucleated egg, wherein the enucleated egg receiving the nucleus develops into a progenitor of the female fish comprising the homozygous alteration.

10. The method of claim 8, wherein the gene-editing comprises microinjection, lipid-based transfection, chemical-based transfection, electroporation, viral-mediated transduction, or exosome-mediated transfected, and a combination thereof.

11. The method of claim 1, wherein the progenitor precedes the female fish by at least one generation, at least two generations, at least three generations, at least five generations, at least ten generations, or at least one hundred generations.

12. The method of claim 10, wherein the gene-editing comprises use of a nuclease.

13. The method of claim 10, wherein the gene-editing comprises a site-specific gene editing system which comprises CRISPR/CaS, a TALEN, a zinc finger nuclease, or a meganuclease.

14. The method of claim 13, wherein the gene editing system creates an alteration that comprises a deletion in the 3′-UTR or an insertion of a nucleic acid sequence into the 3′-UTR.

15. The method of claim 14, wherein the deletion or insertion creates a premature truncation of the wild-type 3′-UTR that prevents normal development and/or normal migration of primordial germ cells, e.g., by reducing or abolishing recognition of the 3′-UTR by its binding protein.

16. The method of claim 15, wherein the gene-editing comprises a polynucleotide.

17. The method of claim 16, wherein the polynucleotide comprises one or more regions homologous to the gene's 3′-UTR nucleotide sequence and/or one or more regions non-homologous to the gene's 3′-UTR nucleotide sequence.

18. The method of claim 17, wherein the polynucleotide comprises a homology directed repair (HDR) template or the polynucleotide comprises a guide RNA (gRNA).

19. The method of claim 18, wherein the gene-editing comprises a polynucleotide and a guide RNA (gRNA).

20. The method of claim 19, wherein the non-homologous region comprises a sequence that prevents normal development and/or normal migration of primordial germ cells.

21. The method of claim 20, wherein the non-homologous region comprises a coding sequence for an exogenous gene.

22. The method of claim 21, wherein the exogenous gene encodes a fluorescent protein, e.g., a derivative or variant of green-fluorescent protein (GFP).

23. The method of claim 1, wherein the sterile fish is selected from tilapia, salmon, trout, tuna, seabass, bream, seabream, barramundi, milkfish, catla, carp, catfish, amberjack, and zebrafish.

24. The method of claim 1, wherein the female fish comprising a gene-edited homozygous alteration further comprises an improved trait relative to a wild-type fish of similar species.

25. The method of claim 24, wherein the improved trait is one or more of area of fat depot, body shape, disease resistance, faster growth, fat percentage, flesh color, greater protein content, improved fertility, larger muscles, skin color, and temperature tolerance.

26. A sterile fish obtained by the method of any one of claims 1 to 25.

27. A cell comprising a heterozygous alteration or homozygous alteration in the three prime untranslated region (3′-UTR) of a gene, wherein the gene contributes to normal development and/or normal migration of primordial germ cells, wherein the gene is selected from nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, and puf/puf-A.

28. A fish comprising a heterozygous alteration or homozygous alteration in the three prime untranslated region (3′-UTR) of a gene, wherein the gene contributes to normal development and/or normal migration of primordial germ cells, wherein the gene is selected from nanos3/nanos/nanos1, dnd1/dnd, ddx4/vasa, dazl, tdrd7, grip2, CaOC1q, cxcr4/cxcr4b, ly75, nlk1, nanog, cpsf6/CFlm68, cxcl12/sdf1, kop, piwi/ziwi, oct4, bucky ball, cxcr7, granulito, hub, miR-430, mkif5Ba, oskar, and puf/puf-A.

29. The fish of claim 28, wherein the fish is selected from tilapia, salmon, trout, tuna, seabass, bream, seabream, barramundi, milkfish, catla, carp, catfish, amberjack, and zebrafish.

30. The fish of claim 29, wherein the fish further comprises an improved trait relative to a wild-type fish of similar species and is one or more of area of fat depot, body shape, disease resistance, faster growth, fat percentage, flesh color, greater protein content, improved fertility, larger muscles, skin color, and temperature tolerance.
